(19) United States
(12) Patent Application Publication
(10) Pub. No.: US 2021/0205223 A1
(43) Pub. Date: Jul. 8, 2021

(54) PROPELLANT-FREE FORMULATION FOR INHALATION

(71) Applicants: Cai Gu Huang, Shrewsbury, MA (US); Ning He, Shanghai (CN); Peng Peng Gu, Shanghai (CN)

(72) Inventors: Cai Gu Huang, Shrewsbury, MA (US); Ning He, Shanghai (CN); Peng Peng Gu, Shanghai (CN)

(73) Assignee: Cai Gu Huang, Shrewsbury, MA (US)

(21) Appl. No.: 17/137,559

(22) Filed: Dec. 30, 2020

Related U.S. Application Data
(60) Provisional application No. 62/957,207, filed on Jan. 4, 2020.

(51) Int. Cl. A61K 9/16; A61K 31/165; A61K 31/40

(52) U.S. Cl. CPC A61K 9/1617; A61K 31/40; A61K 31/165

(57) ABSTRACT

The present invention provides a propellant-free pharmaceutical formulation and a method for administering the pharmaceutical formulation by nebulizing the pharmaceutical formulation in an inhaler. The propellant-free pharmaceutical formulation comprises: (a) glycopyrronium or a salt thereof; (b) formoterol or a salt thereof; (c) a pharmacologically acceptable stabilizer; (d) a pharmacologically acceptable preservative; and (d) a solvent. Additionally, the present invention provides the use of a combination product comprising glycopyrronium or a salt thereof and formoterol or a salt thereof for the prevention or treatment of chronic obstructive pulmonary disease (COPD) and other respiratory diseases.

### PRIORITY STATEMENT

This application claims the benefit of the filing date of U.S. Provisional Patent Application No. 62/957,207, filed on Jan. 4, 2020, the contents of which are incorporated herein by reference in its entirety.

### FIELD OF THE INVENTION

The present invention relates to pharmaceutical formulations containing glycopyrronium or a salt thereof and formoterol or a salt thereof.

### BACKGROUND OF THE INVENTION

Glycopyrronium bromide, also known as glycopyrrolate, is a long acting anti-muscarinic agent (LAMA) and anticholinergic agent, which has the following chemical structure:

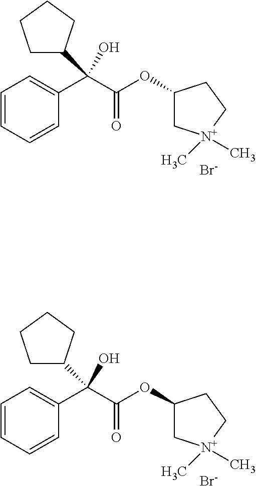 [SMILES: C[N+]1(C)CC[C@@H](OC(=O)[C@@](O)(c2ccccc2)C2CCCC2)C1 ; [Br-] ; C[N+]1(C)CC[C@H](OC(=O)[C@](O)(c2ccccc2)C2CCCC2)C1 ; [Br-]]

Glycopyrronium bromide is commercially available, and the synthesis has been described in U.S. Pat. No. 2,956,062. It is particularly active as an antagonist on the M3 sub-type cholinergic receptors, and is used to reduce salivation associated with administration of certain anesthetics. Glycopyrronium bromide does not cross the blood brain barrier and it penetrates biological membranes slowly, which therefore leads to very few side effects.

Formoterol, chemically known as N-[2-hydroxy-5-(1-hydroxy-2-((2-(4-methoxyphenyl)-1-methylethy-1) amino)-ethyl) phenyl] formamide, has been described in U.S. Pat. No. 3,994,974. Formoterol is typically available as formoterol fumarate, which has the following chemical structure:

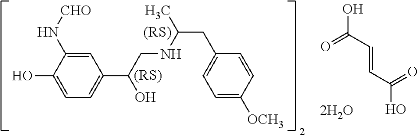

Formoterol fumarate is a long-acting beta 2-adrenergic receptor agonist, is a bronchodilator, and is used in the treatment of obstructive airways diseases. It can be used to treat asthma, shortness of breath, and breathing difficulties caused by chronic obstructive pulmonary disease, as well as a group of lung diseases including chronic bronchitis and emphysema in adults. Inhaled formoterol fumarate acts locally in the lungs to expand the airways. Both formoterol fumarate and glycopyrronium bromide can provide therapeutic benefits for the treatment of asthma and chronic obstructive pulmonary disease.

Glycopyrronium bromide and formoterol fumarate formulations which are administered by a pressurized metered dose inhaler (pMDI) have been developed. However, for a metered-dose inhaler, a propellant, such as hydrofluoroalkane (HFA), is used, which can cause the inhaler to be more expensive. Moreover, these inhaler devices are inefficient and may be difficult or cumbersome to use. Some of the limitations of these inhaler devices are accentuated in patients with chronic obstructive pulmonary disease (COPD), especially for patients who are elderly or have severe disease.

The present invention provides a propellant-free inhalable formulation of formoterol or a salt thereof and glycopyrronium or a salt thereof dissolved in a solvent, such as water, or mixture of solvents, preferably administered by a soft mist inhalation or nebulization inhalation device, and the propellant-free inhalable aerosols resulting therefrom. The pharmaceutical formulations disclosed in the present invention are especially suitable for soft mist inhalation or nebulization inhalation, which have much better lung deposition, typically up to 55-60%.

In one embodiment, the formoterol or salt thereof is formoterol fumarate and the glycopyrronium or salt thereof is glycopyrronium bromide.

The pharmaceutical formulations of the present invention are particularly suitable for administering the active substances by the soft mist or nebulization inhalation, and are especially suitable for treating asthma and chronic obstructive pulmonary disease.

### SUMMARY OF THE INVENTION

Accordingly, in one aspect, the present invention is to provide novel pharmaceutical formulations comprising a solution of glycopyrronium bromide and formoterol fumarate. The pharmaceutical formulation comprises: (a) glycopyrronium bromide; (b) formoterol fumarate; (c) a pharmacologically acceptable stabilizer; (d) a pharmacologically acceptable preservative; and (e) a solvent.

In another aspect, the present invention provides novel propellant-free pharmaceutical formulations which are preferably suitable for use with soft mist inhalers or nebulization inhalers.

In another aspect, the present invention provides the use of a combination product comprising glycopyrronium bromide and formoterol fumarate for the prevention or treatment of chronic obstructive pulmonary disease and other respiratory diseases.

In yet another aspect, the present invention provides methods for the prevention or treatment of chronic obstructive pulmonary disease and other respiratory diseases by administering the pharmaceutical formulation to a subject in need thereof.

### BRIEF DESCRIPTION OF THE DRAWINGS

FIG. 1 is a longitudinal section through an atomizer in the stressed state;

FIG. 2 is a counter element of the atomizer;

FIG. 3 is a graph of the particle size distribution of glycopyrronium bromide of example 4;

FIG. 4 is a graph of the particle size distribution of formoterol fumarate of example 4; and

FIG. 5 is a graph of the particle size distribution of droplets sprayed by soft mist inhaler.

### DETAILED DESCRIPTION OF THE INVENTION

It is advantageous to administer a liquid formulation of an active substance(s) without propellant gases using suitable inhalers, to achieve a better distribution of the active substance(s) in the lung. Furthermore, it is desirable to maximize the lung deposition of the drug delivered by inhalation.

Traditional pMDI or drying powder inhalation (DPI) can only deliver about 20-30% of the drug into the lung, resulting in a significant amount of drug being deposited on the mouth and throat, which can then go to the stomach and cause unwanted side effects and/or secondary absorption through the oral digestive system.

Therefore, there is a need to significantly increase lung deposition when administering a drug by inhalation delivery. The soft mist inhalation device disclosed in U.S. 2019/0030268 can significantly increase the lung deposition of inhalable drugs.

These inhalers nebulize a small amount of a liquid formulation within a few seconds to provide an aerosol that is suitable for therapeutic inhalation. The inhalers are particularly suitable for use with the liquid formulations disclosed herein.

Soft mist devices suitable for use with the pharmaceutical formulations of the present invention are those in which an amount of less than about 70 microliters of pharmaceutical solution can be nebulized in one puff, such as less than about 30 microliters, or less than about 15 microliters, so that the inhalable part of aerosol corresponds to the therapeutically effective quantity. The average particle size of the aerosol formed from one puff is less than about 15 microns, such as less than about 10 microns.

A device of this kind for the propellant-free administration of a metered amount of a liquid pharmaceutical formulation for inhalation is described in detail, for example, in U.S. 2019/0030268, entitled “Inhalation atomizer comprising a blocking function and a counter”.

The pharmaceutical formulation in the soft mist devices is converted into an aerosol destined for the lungs. The soft mist device uses high pressure to spray the pharmaceutical formulation.

The pharmaceutical formulation of the invention is stored in a reservoir in these kinds of inhalers. The pharmaceutical formulation must not contain any ingredients which might interact with the inhaler and affect the pharmaceutical quality of the solution or of the aerosol produced. In addition, it is desirable that the active substances in pharmaceutical formulations are very stable when stored and are capable of being administered directly.

The pharmaceutical formulation of the present invention for the inhaler described above preferably contain additives, such as the disodium salt of edetic acid (sodium edetate), to reduce the incidence of spray anomalies and to stabilize the solutions. The pharmaceutical formulation of the invention preferably has a minimum concentration of sodium edetate.

Therefore, one aspect of the present invention is to provide novel pharmaceutical solution formulations comprising glycopyrronium bromide and formoterol fumarate, which meet the high standards needed to achieve optimum nebulization of the solution using the inhalers mentioned hereinbefore. In an embodiment, the active substances in the pharmaceutical formulation are stable and have a storage time of some years, such as about one year, or, for example, about three years.

In another aspect, the present invention provides novel propellant-free pharmaceutical solution formulations which are preferably suitable for use with soft mist inhalers.

In another aspect, the present invention provides the use of a combination product comprising glycopyrronium bromide and formoterol fumarate for the prevention or treatment of chronic obstructive pulmonary disease and other respiratory diseases.

In yet another aspect, the present invention provides methods for preventing or treating chronic obstructive pulmonary disease and other respiratory diseases by administering the formulations to a subject in need thereof.

According to the invention, any pharmaceutically acceptable salts or solvates of glycopyrronium and formoterol may be used in the formulations. In one aspect of the invention, the salt or solvate of glycopyrronium is glycopyrronium bromide, and the salt or solvate of formoterol is formoterol fumarate.

In an embodiment, the active substances are glycopyrronium bromide and formoterol fumarate in combination.

The concentration of the glycopyrronium bromide and formoterol fumarate in the finished pharmaceutical formulation depends on the therapeutic effects.

The glycopyrronium component of the formulation can be in the form of the free base, or as a salt or a solvate. In an embodiment, the glycopyrronium is provided in the form of glycopyrronium bromide. Glycopyrronium bromide is typically present in the formulation in an amount in the range from about 30 mg/100 g to about 300 mg/100 g, preferably from about 50 mg/100 g to about 200 mg/100 g.

The formoterol component of the formulation can be in the form of the free base, or as a salt or a solvate. In an embodiment, the formoterol is provided in the form of formoterol fumarate. Formoterol fumarate can, for instance, be employed in the formulation in an amount of from about 10 mg/100 g to about 100 mg/100 g, preferably from about 20 mg/100 g to about 70 mg/100 g.

As used herein, the term “formoterol fumarate” refers to the salt in which formoterol can be each of the possible isomers either in substantially pure form or admixed in any proportions, preferably as a racemic mixture of the (R, R) and (S, S) stereoisomers.

As used herein, the term “mass median aerodynamic diameter” means the diameter of 50 percent by weight of the aerosolized particles upon actuation of the inhaler.

In an embodiment, glycopyrronium bromide is present in the formulation in an amount of about 100 mg/100 g, and formoterol fumarate is present in the formulation in an amount of about 50 mg/100 g.

In the formulations according to the invention, the glycopyrronium bromide and formoterol fumarate are dissolved in a solvent. In one embodiment, the solvent is water or water-ethanol co-solvents.

In one embodiment, the formulation further comprises a stabilizer or complexing agent. In one embodiment the stabilizer or complexing agent is edetic acid (EDTA) or one of the known salts thereof, for example, edetate disodium or edetate disodium dihydrate.

A complexing agent is a molecule which is capable of entering into complex bonds. In an embodiment, these compounds have the effect of complexing cations.

Other comparable stabilizers or complexing agents can be used in the present invention. Other stabilizers or complexing agents include, but are not limited to, citric acid and anhydrous citric acid.

In an embodiment, the formulation contains edetic acid and/or the salts thereof.

The concentration of the stabilizer or complexing agents is typically about 5.0 mg/100 g to about 22 mg/100 g. In an embodiment, the concentration of the stabilizer or complexing agents is about 5.0 mg/100 g to about 20.0 mg/100 g.

In one embodiment, the concentration of edetate disodium dihydrate is about 5 mg/100 g to about 40 mg/100 g, such as from about 8 mg/100 g to about 20 mg/100 g.

In one embodiment, the concentration of edetate disodium dihydrate is about 11 mg/100 g.

In an embodiment, the glycopyrronium bromide and formoterol fumarate are present in solution.

In an embodiment, all the ingredients of the formulation are present in solution.

In an embodiment, the formulation of the present invention comprises a solvent. In one embodiment, the solvent is selected from water and normal saline. In one embodiment, the solvent is water.

The solvent may be present in the formulation in amounts of about 50 wt % to about 99.8 wt %.

In one embodiment, the solvent is present in the formulation in an amount of about 99.7 wt %.

If desired, other co-solvents may be added to the formulation according to the invention. In an embodiment, other co-solvents are those which contain hydroxyl groups or other polar groups, such as, alcohols, isopropyl alcohol, propylene glycol, polyethylene glycol, polypropylene glycol, glycerol, polyoxyethylene alcohols.

The formulations may further comprise additives. The term “additives,” as used herein means any pharmacologically acceptable and therapeutically useful substance which is not an active substance, but which can be formulated together with the active substances in the solvent, to improve the qualities of the formulation.

In an embodiment, these additives have no appreciable pharmacological effects or at least no undesirable pharmacological effects in the context of the desired therapy.

The additives include, but are not limited to, for example, other stabilizers, complexing agents, antioxidants, surfactants, and/or preservatives which prolong the shelf life of the finished pharmaceutical formulation, vitamins and/or other additives known in the art.

In one aspect of the invention, the formulations further comprise a suitable preservative to protect the formulation from contamination with pathogenic bacteria. In one embodiment, the preservative comprises benzalkonium chloride, benzoic acid, or sodium benzoate. In one embodiment, the formulations contain 50% benzalkonium chloride.

As used herein, “preservative” refers to a compound that is added to a pharmaceutical formulation to act as an antimicrobial. Suitable preservatives include, but are not limited to, for example, benzethonium, benzalkonium chloride, chlorohexidine, phenol, m-cresol, benzyl alcohol, methylparaben, propylparaben, chlorobutanol, o-cresol, p-cresol, chlorocresol, benzalconium chloride, phenylmercuric nitrate, thimerosal, and benzoic acid. In an embodiment, the formulation includes a single type of preservative. If desired, a combination of two or more types of preservative may be used.

The preservative is typically present in the formulation in an amount from about 10 mg/100 g to about 50 mg/100 g, such as in an amount from about 20 mg/100 g to about 30 mg/100 g.

In an embodiment, the content of benzalkonium chloride is about 20 mg/100 g to about 30 mg/100 g.

As used herein, the term “pH buffering agent” or “buffer” refers to a component that improves isotonicity and chemical stability of the formulation, and functions to maintain a physiologically suitable pH. The buffer maintains the pH of the solution to provide better stabilization of the active components.

In one aspect of the invention, the formulation comprises a pH buffering agent. In one embodiment, the pH buffering agent is selected from the group consisting of anhydrous citric acid, ascorbic acid, citric acid, malic acid, tartaric acid, maleic acid, fumaric acid, acetic acid, and/or propionic acid, etc.

In one embodiment, the pH buffering agent is selected from anhydrous citric acid.

If desired, inorganic acids, for example, hydrochloric acid, hydrobromic acid, nitric acid, sulfuric acid, and/or phosphoric acid, may also be used. In one embodiment, hydrochloric acid is used.

The formulation of the present invention may comprise a pH adjusting agent, such as a pharmacologically acceptable base. Pharmacologically acceptable bases include, but are not limited to, alkali metal hydroxides and alkali metal carbonates. The preferred alkali metal ion is sodium. If bases of this kind are used, it should be ensured that the resulting salts, which are then contained in the finished pharmaceutical formulation, are pharmacologically compatible with the abovementioned acid.

In one embodiment, the pH adjusting agent is sodium hydroxide.

In an embodiment, the pH adjusting agent is present in the formulation in an amount from about 60 mg/100 g to about 120 mg/100 g.

In one embodiment, the pH adjusting agent is present in the formulation in an amount of about 96 mg/100 g.

In the formulations according to the invention, the pH of the formulation is between about 2.5 and about 7.5, preferably between about 3.5 and about 6.0, or more preferably between about 4.0 and about 5.5.

In one embodiment, the pH of the formulation is adjusted with a pH adjusting agent to a pH of about 5.0.

The formulation according to the present invention may be administered orally or via inhalation.

To produce the propellant-free aerosols according to the invention, the pharmaceutical formulations containing glycopyrronium bromide and formoterol fumarate are typically used with an inhaler of the kind described hereinbefore.

The inhaler disclosed in U.S. 2019/0030268 is an example of an inhaler that is suitable for use with the formulations of the present invention. This soft mist nebulizer can be used to produce the inhalable aerosols according to the invention.

The inhalable device can be carried anywhere by the patient, since it has a cylindrical shape and handy size of less than about 8 cm to about 18 cm long, and about 2.5 cm to about 5 cm wide. The nebulizer sprays out a defined volume of the pharmaceutical formulation through small nozzles at high pressure, so as to produce an inhalable aerosol.

The preferred atomizer comprises an atomizer 1, a fluid 2, a vessel 3, a fluid compartment 4, a pressure generator 5, a holder 6, a drive spring 7, a delivering tube 9, a non-return valve 10, pressure room 11, a nozzle 12, a mouthpiece 13, an aerosol 14, an air inlet 15, an upper shell 16, an inside part 17.

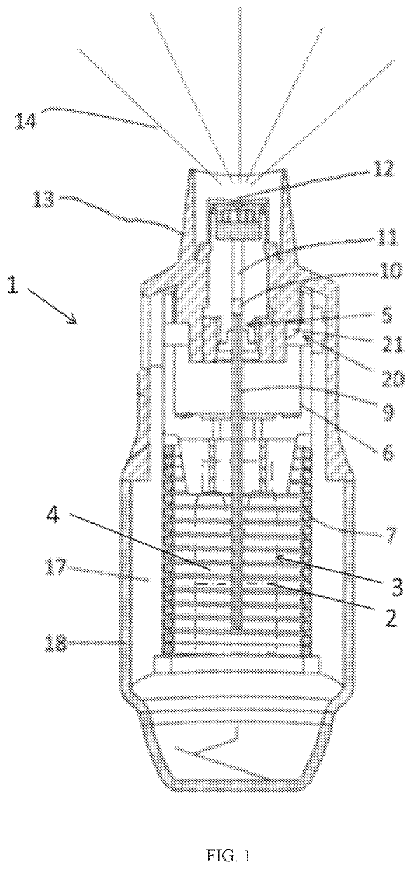

The inhalation atomizer 1 comprising the block function and the counter described above for spraying a medicament fluid 2 is depicted in the FIG. 1 in a stressed state. The atomizer 1 comprising the block function and the counter described above is preferably a portable inhaler and propellant-free.

For the typical atomizer 1 comprising the block function and the counter described above, an aerosol 14 that can be inhaled by a patient is generated through atomization of the fluid 2, which is typically formulated as a medicament liquid. The medicament is typically administered at least once a day, more specifically multiple times a day, preferably at predestined time gaps, according to how serious the illness affects the patient.

In an embodiment, the atomizer 1 described above has a substitutable and insertable vessel 3, which contains the medicament fluid 2. Therefore, a reservoir for holding the fluid 2 is formed in the vessel 3. Specifically, the medicament fluid 2 is located in the fluid compartment 4 formed by a collapsible bag in the vessel 3.

In an embodiment, the amount of fluid 2 for the inhalation atomizer 1 described above is in the vessel 3 to provide, for example, up to 200 doses. A classical vessel 3 has a volume of about 2 ml to about 10 ml. A pressure generator 5 in the atomizer 1 is used to deliver and atomize the fluid 2, in a pre-determined dosage amount. Therefore, the fluid 2 is released and sprayed in individual doses, specifically doses of from about 5 to about 30 microliters.

In an embodiment, the atomizer 1 described above may have a pressure generator 5, a holder 6, a drive spring 7, a delivering tube 9, a non-return valve 10, a pressure room 11, and a nozzle 12 in the area of a mouthpiece 13. The vessel 3 is latched by the holder 6 in the atomizer 1 so that the delivering tube 9 is plunged into the vessel 3. The vessel 3 can be separated from the atomizer 1 for substitution.

In an embodiment, when drive spring 7 is stressed in an axial direction, the delivering tube 9, the vessel 3 along with the holder 6 will be shifted downwards. Then the fluid 2 will be sucked into the pressure room 11 through delivering tube 9 and non-return valve 10.

In an embodiment, after releasing the holder 6, the stress is eased. During this process, the delivering tube 9 and closed non-return valve 10 are shifted upward by releasing the drive spring 7. Consequently, the fluid 2 is under the pressure in the pressure room 11. The fluid 2 is then pushed through the nozzle 12 and atomized into an aerosol 14 by the resulting pressure. A patient can inhale the aerosol 14 through the mouthpiece 13, while the air is sucked into the mouthpiece 13 through air inlets 15.

In an embodiment, the atomizer 1 comprising the block function and the counter described above has an upper shell 16 and an inside part 17, which can be rotated relative to the upper shell 16. A lower shell 18 is manually operable to attach onto the inside part 17. The lower shell 18 can be separated from the atomizer 1 so that the vessel 3 can be substituted and inserted.

In an embodiment, the inhalation atomizer 1 described above may have a lower shell 18, which carries the inside part 17, being rotatable relative to the upper shell 16. As a result of rotation and engagement between the upper unit 17 and the holder 6, through a gear 20, the holder 6 axially moves the counter in response to the force of the drive spring 7 and the drive spring 7 is stressed.

In an embodiment, in the stressed state the vessel 3 is shifted downwards until it reaches a final position, which is demonstrated in FIG. 1. The drive spring 7 is stressed in this final position. Then the holder 6 is clasped. Therefore, the vessel 3 and the delivering tube 9 are prevented from moving upwards so that the drive spring 7 is stopped from easing.

In an embodiment, the atomizing process occurs after the holder 6 is released. The vessel 3, the delivering tube 9 and the holder 6 are shifted back by the drive spring 7 to the beginning position. This is referred to herein as major shifting. While the major shifting occurs, the non-return valve 10 is closed and the fluid 2 is under pressure in the pressure room 11 by the delivering tube 9, and then the fluid 2 is pushed out and atomized by the pressure.

In an embodiment, the inhalation atomizer 1 described above may have a clamping function. During the clamping, the vessel 3 preferably performs a lifting shift for the withdrawal of the fluid 2 during the atomizing process. The gear 20 has sliding surfaces 21 on the upper shell 16 and/or on the holder 6, which could make holder 6 axially move when the holder 6 is rotated relative to the upper shell 16.

In an embodiment, the holder 6 is not blocked for too long and can carry on the major shifting. Therefore, the fluid 2 is pushed out and atomized.

In an embodiment, when the holder 6 is in the clamping position, the sliding surfaces 21 move out of engagement. Then the gear 20 releases the holder 6 for the opposite shift axially.

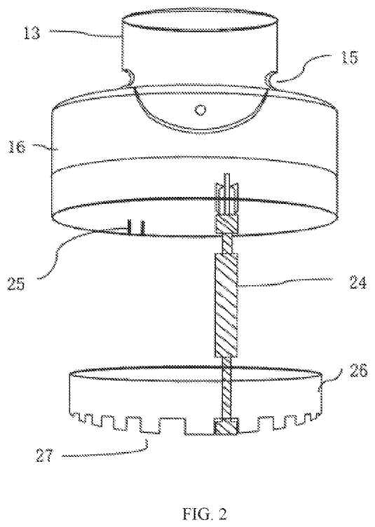

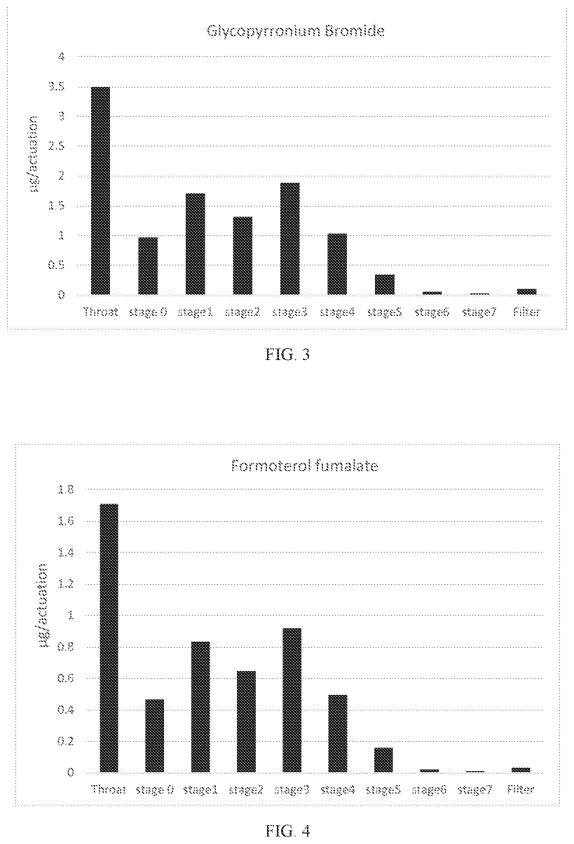

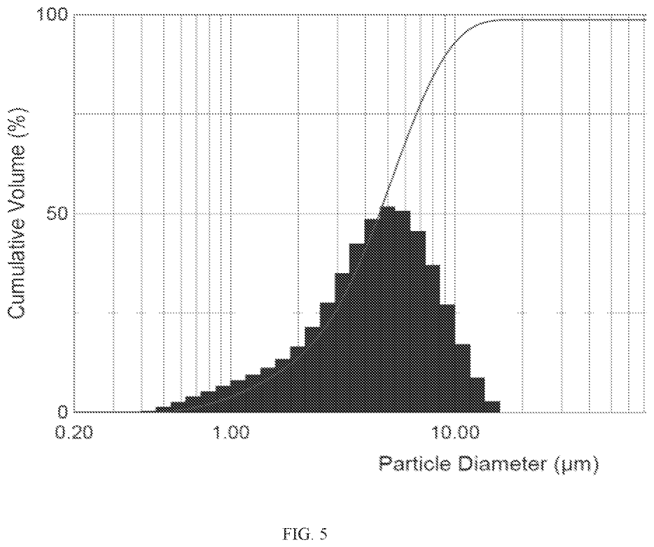

In an embodiment, the atomizer 1 preferably includes a counter element shown in FIG. 2. The counter element has a worm 24 and a counter ring 26. The counter ring 26 is typically circular and has dentate part at the bottom. The worm 24 has upper and lower end gears. The upper end gear contacts with the upper shell 16. The upper shell 16 has inside bulge 25. When the atomizer 1 is employed, the upper shell 16 rotates; and when the bulge 25 passes through the upper end gear of the worm 24, the worm 24 is driven to rotate. The rotation of the worm 24 drives the rotation of the counter ring 26 through the lower end gear so as to result in a counting effect.

In an embodiment, the locking mechanism is realized mainly by two protrusions. Protrusion A is located on the outer wall of the lower unit of the inside part. Protrusion B is located on the inner wall of counter. The lower unit of the inside part is nested in the counter. The counter can rotate relative to the lower unit of the inside part. Because of the rotation of the counter, the number displayed on the counter can change as the actuation number increases, and can be observed by the patient. After each actuation, the number displayed on the counter changes. Once the predetermined number of actuations is achieved, Protrusion A and Protrusion B will encounter each other and the counter will be prevented from further rotation. This blocks the atomizer, stopping it from further use. The number of actuations of the device can be counted by the counter.

The nebulizer described above is suitable for nebulizing the aerosol preparations according to the invention to form an aerosol suitable for inhalation. Nevertheless, the formulation according to the invention can also be nebulized using other inhalers apart from those described above, such as an ultrasonic vibrating mesh nebulizers or compressed air nebulizers.

The following examples are intended to illustrate and exemplify the various aspects of carrying out the present invention and are not intended to limit the invention in any way.

### EXAMPLES

Materials and Reagents:

50% benzalkonium chloride is commercially available and may be purchased from Spectrum Pharmaceuticals Inc.

Glycopyrronium bromide is also commercially available and may be purchased from Nanjing Daqin Pharma Co., Ltd.

Edetate disodium dihydrate is also commercially available and may be purchased from Nanjing Reagent Co., Ltd.

Formoterol fumarate is also commercially available and may be purchased from Hubei Weideli Chemical Tech Co., Ltd.

### Example 1

The preparation of the formulation of sample 1 is described below:

50% benzalkonium chloride (50% BAC) in the amount provided in table 1 was dissolved in purified water and then transferred into a beaker. Edetate disodium dihydrate and anhydrous citric acid in the amounts provided in table 1 were added to the solution, and then 95 g of purified water was added thereto, and the resulting mixture was sonicated until completely dissolved. Thereafter, the pH was adjusted to 5.0 by the addition of 1M sodium hydroxide solution. Formoterol fumarate in the amount provided in table 1 was added to the solution, and the resulting mixture was sonicated until completely dissolved. After complete dissolution, glycopyrronium bromide was added to the solution, and the resulting mixture was sonicated until completely dissolved. The final total weight of the solution was 100 g.

TABLE 1
Ingredient contents of sample 1 of 100 g inhalable formulation

| Ingredients | Sample 1 | |
|---|---|---|
| Formoterol fumarate | 50 | mg |
| Glycopyrronium bromide | 100 | mg |
| Edetate disodium dihydrate | 11 | mg |
| 50% benzalkonium chloride | 20 | mg |
| Anhydrous citric acid | 96 | mg |

| | |
|---|---|
| pH adjusted with 1M NaOH | 5.0 |
| Purified water | added to 100 g |

### Example 2

The preparation of the formulation of sample 2 is described below:

50% benzalkonium chloride (50% BAC) in the amount provided in table 2 was dissolved in purified water and then transferred into a beaker. Edetate disodium dihydrate and anhydrous citric acid in the amounts provided in table 2 were added to the solution, and then 95 g of purified water was added thereto, and the resulting mixture was sonicated until completely dissolved. Thereafter, the pH was adjusted to 4.8 by the addition of 1M sodium hydroxide solution. Formoterol fumarate in the amount provided in table 2 was added to the solution, and the resulting mixture was sonicated until completely dissolved. After complete dissolution, glycopyrronium bromide was added to the solution, and the resulting mixture was sonicated until completely dissolved. The final total weight of mixed solution was 100 g.

TABLE 2
Ingredient contents of sample 1 of 100 g inhalable formulation

| Ingredients | Sample 2 | |
|---|---|---|
| Formoterol fumarate | 50 | mg |
| Glycopyrronium bromide | 100 | mg |
| Edetate disodium dihydrate | 11 | mg |
| 50% benzalkonium chloride | 20 | mg |
| Anhydrous citric acid | 96 | mg |

| | |
|---|---|
| pH adjusted with 1M NaOH | 4.8 |
| Purified water | added to 100 g |

### Example 3

The preparation of the formulation of sample 3 is described below:

50% benzalkonium chloride (50% BAC) in the amount provided in table 3 was dissolved in purified water and then transferred into a beaker. Edetate disodium dihydrate and anhydrous citric acid in the amounts provided in table 3 were added to the solution, and then 95 g of purified water was added thereto, and the resulting mixture was sonicated until completely dissolved. Thereafter, the pH was adjusted to 5.2 by the addition of 1M sodium hydroxide solution. Formoterol fumarate in the amount provided in table 3 was added to the solution, and the resulting mixture was sonicated until completely dissolved. After complete dissolution, glycopyrronium bromide was added to the solution, and the resulting mixture was sonicated until completely dissolved. The final total weight of mixed solution was 100 g.

TABLE 3
Ingredient contents of sample 1 of 100 g inhalable formulation

| Ingredients | Sample 3 | |
|---|---|---|
| Formoterol fumarate | 50 | mg |
| Glycopyrronium bromide | 100 | mg |
| Edetate disodium dihydrate | 11 | mg |
| 50% benzalkonium chloride | 20 | mg |
| Anhydrous citric acid | 96 | mg |

| | |
|---|---|
| pH adjusted with 1M NaOH | 5.2 |
| Purified water | added to 100 g |

### Example 4

Aerodynamic Particle Size Distribution:

The aerodynamic particle size distribution was determined using an Andersen Scale Impactor (ACI). The soft mist inhaler was held close to the ACI inlet until no aerosol was visible. The flow rate of the ACI was set to 28.3 L/minute and was operated under ambient temperature and a relative humidity (RH) of 90%.

The solution of sample 1 was discharged into the ACI. Fractions of the dose were deposited at different stages of the ACI, in accordance with the particle size of the fraction. Each fraction was washed from the stage and analyzed using HPLC.

The particle size distribution was expressed in terms of mass median aerodynamic diameter (MMAD) and Geometric Standard Deviation (GSD). The results showed that the MMAD of glycopyrronium bromide and formoterol fumarate were less than 6 μm, and the GSD of both glycopyrronium bromide and formoterol fumarate were less than 2. The results are provided in Table 4 below.

TABLE 4
Aerodynamic particle size distribution

| Particle size parameter | Formoterol fumarate | Glycopyrronium bromide |
|---|---|---|
| MMAD (μm) | 4.49 | 4.91 |
| GSD | 1.58 | 1.59 |

### Example 5

Sample 1 was sprayed using a soft mist inhaler. A Malvern Spraytec (STP5311) was used to measure particle size distribution of droplets sprayed by the soft mist inhaler. As shown in Table 5, the results indicated that the D50 from sample 1 was less than 5 μm, and the D90 from sample 1 was less than 10 μm. The particle size distribution of droplets sprayed by the soft mist inhaler was uniform.

TABLE 5
Particle size distribution by using soft mist inhaler

| Sample Number | Particle size (μm) | Using soft mist inhaler |
|---|---|---|
| Sample 1 | D₁₀ | 1.625 |
| | D₅₀ | 4.604 |
| | D₉₀ | 9.134 |

### Example 6

To examine the stability of the formulations prepared as described in table 1 (sample 1), table 2 (sample 2) and table 3 (sample 3), the formulations were stored under conditions of 25° C. for 3 months. The pH, assay, and impurities were measured initially, at 1 month, at 2 months, and at 3 months. The remaining amounts of both formoterol fumarate and glycopyrronium bromide at each time point were measured by high performance liquid chromatography (HPLC).

As can be seen from Tables 6 to 11, the pharmaceutical formulation according to the invention shows excellent stability with no significant variation in pH or amount of the active components.

TABLE 6
Stability of sample 1
Stability of formoterol fumarate under conditions: 25° C.

| Test | Initial | 1 month | 2 months | 3 months |
|---|---|---|---|---|
| pH | 5.0 | 5.03 | 5.02 | 5.04 |
| Assay (mg/100 g) | 51.64 | 47.77 | 51.19 | 50.37 |
| Unknown maximum | 0.11 | 0.52 | 0.90 | 1.02 |
| impurity (%) | | | | |
| Total impurities (%) | 0.15 | 0.78 | 1.82 | 1.71 |

TABLE 7
Stability of sample 1
Stability of glycopyrronium bromide under conditions: 25° C.

| Test | Initial | 1 month | 2 months | 3 months |
|---|---|---|---|---|
| pH | 5.0 | 5.03 | 5.02 | 5.04 |
| Assay (mg/100 g) | 10.28 | 10.38 | 10.22 | 10.30 |
| Degradant J of GB | ND | ND | 0.57 | 0.80 |
| (%) | | | | |
| Total impurities (%) | ND | ND | 0.57 | 0.80 |

“ND”: Not detected; Degradant J is 2-cyclopenty-2-hydroxy-2-phenylacetic acid

TABLE 8
Stability of sample 2
Stability of formoterol fumarate under conditions: 25° C.

| Test | Initial | 1 month | 2 months | 3 months |
|---|---|---|---|---|
| pH | 4.8 | 4.88 | 4.88 | 4.89 |
| Assay (mg/100 g) | 51.54 | 50.80 | 51.59 | 50.51 |
| Unknown maximum | 0.12 | 0.58 | 1.01 | 1.16 |
| impurity (%) | | | | |
| Total impurities (%) | 0.19 | 0.90 | 1.97 | 1.92 |

TABLE 9
Stability of sample 2
Stability of glycopyrronium bromide under conditions: 25° C.

| Test | Initial | 1 month | 2 months | 3 months |
|---|---|---|---|---|
| pH | 4.8 | 4.88 | 4.88 | 4.89 |
| Assay (mg/100 g) | 10.14 | 10.22 | 10.21 | 10.27 |
| Degradant J of GB | ND | ND | 0.41 | 0.62 |
| (%) | | | | |
| Total impurities (%) | ND | ND | 0.41 | 0.62 |

“ND”: Not detected.

TABLE 10
Stability of sample 3
Stability of formoterol fumarate under conditions: 25° C.

| Test | Initial | 1 month | 2 months | 3 months |
|---|---|---|---|---|
| pH | 5.2 | 5.24 | 5.25 | 5.25 |
| Assay (mg/100 g) | 50.57 | 50.33 | 51.51 | 50.69 |
| Unknown maximum | 0.10 | 0.46 | 0.74 | 0.81 |
| impurity (%) | | | | |
| Total impurities (%) | 0.18 | 0.75 | 1.50 | 1.54 |

TABLE 11
Stability of sample 3
Stability of glycopyrronium bromide under conditions: 25° C.

| Test | Initial | 1 month | 2 months | 3 months |
|---|---|---|---|---|
| pH | 5.2 | 5.24 | 5.25 | 5.25 |
| Assay (mg/100 g) | 10.00 | 9.90 | 10.10 | 10.18 |
| Degradant J of GB | ND | ND | 0.92 | 1.26 |
| (%) | | | | |
| Total impurities (%) | ND | ND | 0.92 | 1.26 |

“ND”: Not detected.

### Example 7

The preparation of the formulation of sample 4 is described below:

50% benzalkonium chloride (50% BAC) in the amount provided in table 12 was dissolved in purified water and then transferred into a beaker. Edetate disodium dihydrate in the amounts provided in table 12 were added to the solution, and then 49 g of purified water was added thereto, and the resulting mixture was sonicated until completely dissolved. Thereafter, the pH was adjusted to 5.0 by the addition of citric acid solution. Formoterol fumarate in the amount provided in table 12 was added to the solution, and the resulting mixture was sonicated until completely dissolved. After complete dissolution, glycopyrronium bromide was added to the solution, and the resulting mixture was sonicated until completely dissolved. The final total weight of the solution was 50 g.

TABLE 12
Ingredient contents of sample 4 of 50 g inhalable formulation

| Ingredients | Sample 1 | |
|---|---|---|
| Formoterol fumarate | 12.50 | mg |
| Glycopyrronium bromide | 25 | mg |
| Edetate disodium dihydrate | 5 | mg |
| 50% benzalkonium chloride | 10 | mg |

| | |
|---|---|
| Anhydrous citric acid | Adjusting to pH 5.0 |
| Purified water | added to 50 g |

To examine the stability of the formulations prepared as described in table 12 (sample 4), the formulations were stored under conditions of 40° C. for 1 month. The assay and impurities were measured initially, at 5 days, at 10 days, and at 1 month. The remaining amounts of both formoterol fumarate and glycopyrronium bromide at each time point were measured by high performance liquid chromatography (HPLC).

As can be seen from Tables 13 and 14, the pharmaceutical formulation according to the invention shows excellent stability with no significant variation in pH or amount of the active components.

TABLE 13
Stability of sample 4
Stability of formoterol fumarate under conditions: 40° C.

| Test | Initial | 5 days | 10 days | 1 month |
|---|---|---|---|---|
| Assay (mg/100 g) | 25.99 | 24.77 | 24.6 | 24.63 |
| Unknown maximum | 0.172 | 0.407 | 0.763 | 1.62 |
| impurity (%) | | | | |
| Total impurities (%) | 0.172 | 0.425 | 0.878 | 2.077 |

TABLE 14
Stability of sample 4
Stability of glycopyrronium bromide under conditions: 40° C.

| Test | Initial | 5 days | 10 days | 1 month |
|---|---|---|---|---|
| Assay (mg/100 g) | 51.77 | 49.74 | 49.76 | 49.98 |
| Impurity J of GB | ND | 0.175 | 0.341 | 1.193 |
| (%) | | | | |
| Total impurities (%) | ND | 0.175 | 0.341 | 1.193 |

“ND”: Not detected.

What is claimed is:

1. A propellant-free pharmaceutical preparation comprising: (a) glycopyrronium or a salt thereof; (b) formoterol or a salt thereof; (c) a pharmacologically acceptable stabilizer; (d) a pharmacologically acceptable preservative, and (e) a solvent, wherein the pH of the pharmaceutical preparation is between about 2.5 and about 7.5

2. The pharmaceutical preparation according to claim 1, comprising glycopyrronium bromide and formoterol fumarate.

3. The pharmaceutical preparation according to claim 2, wherein the glycopyrronium bromide is present in an amount ranging from about 30 mg/100 g to about 300 mg/100 g.

4. The pharmaceutical preparation according to claim 2, wherein the formoterol fumarate is present in an amount ranging from about 10 mg/100 g to about 100 mg/100 g.

5. The pharmaceutical preparation according to claim 1, wherein the pharmacologically acceptable preservative is selected from the group consisting of benzalkonium chloride, benzoic acid, sodium benzoate, and combinations thereof.

6. The pharmaceutical preparation according to claim 5, wherein the pharmacologically acceptable preservative is present in an amount ranging from about 10 mg/100 g to about 50 mg/100 g.

7. The pharmaceutical preparation according to claim 1, wherein the stabilizer is selected from the group consisting of edetic acid, edetate disodium dehydrate, edetate disodium, and combinations thereof.

8. The pharmaceutical preparation according to claim 1, wherein the stabilizer is present in amount ranging from about 5 mg/100 g to about 22 mg/100 g.

9. The pharmaceutical preparation according to claim 1, wherein the solvent comprises water or normal saline.

10. The pharmaceutical preparation according to claim 9, further comprising a co-solvent containing a hydroxyl group.

11. The pharmaceutical preparation according to claim 10, wherein the co-solvent is selected from the group consisting of isopropyl alcohol, propylene glycol, polyethylene glycol, polypropylene glycol, glycerol, polyoxyethylene alcohols, and combinations thereof.

12. The pharmaceutical preparation according to claim 1, further comprising a pharmacologically acceptable additive.

13. The pharmaceutical preparation according to claim 12, wherein pharmacologically acceptable additive is an antioxidant.

14. A method for administering the pharmaceutical preparation according to claim 1, comprising nebulizing a defined amount of the pharmaceutical preparation through a nozzle by the application of pressure to form an inhalable aerosol.

15. The method according to claim 14, wherein the inhalable aerosol has a mass median aerodynamic diameter (MMAD) of less than about 6 microns.

16. The method according to claim 14, wherein the inhalable aerosol has a particle size distribution with a D50 less than 5 μm.

17. The method of claim 14, wherein the pharmaceutical preparation is nebulized using an inhaler according to FIG. 1.

18. The method according to claim 14, wherein the defined amount of the pharmaceutical preparation ranges from about 5 to about 30 microliters.

19. The method according to claim 17, wherein the inhaler comprises a block function and a counter.

20. A method of treating asthma or COPD in a patient, comprising administering to the patient a pharmaceutical preparation according to claim 1.

21. A method of treating asthma or COPD in a patient, the method comprising administering to the patient a pharmaceutical preparation using the method of claim 14.

22. A method of treating asthma or COPD in a patient, the method comprising administering to the patient a pharmaceutical preparation using the method of claim 17.

23. A method of treating asthma or COPD in a patient, the method comprising administering to the patient a pharmaceutical preparation using the method of claim 19.
